(19) United States
(12) Patent Application Publication
(10) Pub. No.: US 2022/0304743 A1
(43) Pub. Date: Sep. 29, 2022

(54) Skin Tightening Method And Apparatus

(71) Applicant: Intelis Instruments Ltd., Hadera (IL)

(72) Inventor: Boris Vaynberg, Zichron Yakov (IL)

(21) Appl. No.: 17/687,724

(22) Filed: Mar. 7, 2022

Related U.S. Application Data
(60) Provisional application No. 63/164,674, filed on Mar. 23, 2021.

(51) Int. Cl. A61B 18/14

(52) U.S. Cl. CPC A61B 18/1477; A61B 2018/0047

(57) ABSTRACT

The present application discloses a method and apparatus that use a two-dimensional array or matrix of RF electrodes arranged on a substrate. In one example, the RF electrodes are individually addressable. In another example, the RF electrodes are arranged in addressable clusters. The clusters of the RF electrodes could be non-symmetrical dusters of electrodes.

### TECHNOLOGY FIELD

The method and apparatus are related to the field of cosmetic skin treatment and particularly to the field of RF fractal skin tightening.

### BACKGROUND

Skin tightening is a cosmetic procedure to change the look of a person's skin. As a person ages, the skin loses elastin and collagen, which provide the skin with a smooth structure and elasticity. Skin tightening uses heat to stimulate new collagen production and contract existing collagen fibers.

There are several technologies and procedures directed to skin tightening treatment. For example, the application of ultrasound, radiofrequency, intense pulsed light, and laser radiation are just a few of the technologies. Each technology has its advantages, although all of them include a mechanism providing a skin heating effect.

The listed above technologies support nonsurgical skin tightening and apply treatments to skin segments that have become loose to stimulate collagen production underneath that skin. Usually, each of the technologies requires multiple treatment sessions, although some results could be noticed after the first treatment.

The nonsurgical skin tightening procedures are considered low-risk skin treatment procedures. These treatments' side effects are mild and could include some skin swelling, bruising, redness, soreness, and skin burns.

Recently a fractional skin tightening technology has been commercialized. The technology uses a matrix of radiofrequency (RF) electrodes or needles to deliver thermal energy and produce an array of small lesions in the skin. Laser fractal technology uses a plurality of laser beams to produce an array of small lesions in the skin.

The fractional technology has become of use in tightening mild to moderately loose skin on the face, neck, body, and scars masking. Little to no downtime is needed. The fractional skin tightening technology is low-risk and almost does not produce adverse skin effects. Several treatments could be required to produce a noticeable skin tightening effect.

The following patents disclose the current state of the fractional skin treatment technology WO9528886 to Burbank, WO2014059151 to Paliwal, EP1742590 to Ganz, and U.S. Pat. No. 7,087,035 to Troutman.

### SUMMARY

The current document describes a method and apparatus for directional skin tightening. The method includes stretching a segment of the skin in a first direction and applying to the stretched segment of skin at least one needle RF electrode. Force is applied to the skin segment to stretch it into the desired direction. The RF application to the stretched skin segment ablates the skin and creates at least one cylindrical RF ablated skin lesion. The lesion is usually of symmetric shape. Upon the release of the stretch of the stretched skin segment allows the skin to return to its non-stretched or original state. The lesion transforms its symmetrical cross-section shape into an elongated asymmetrical cross-section shape.

The transformation is such that the long axis of the elongated cross-section shape of the cylindrical lesion is perpendicular to the skin stretching direction. The healing of the non-symmetrical (asymmetrical) elongated skin lesion in the direction of short elongated lesion axes is faster than in the direction of a long elongated non-symmetrical lesion axis.

The method supports the addressing of individual RF electrodes configured to generate desired areas of skin lesions. The orientation of the desired areas of skin lesions supports skin tightening in the desired direction.

The apparatus for skin tightening includes a matrix of RF electrodes disposed on a rigid or flexible substrate, an RF generator configured to energize the RF electrodes, and an applicator with a device configured to stretch the treated segment of skin, and a control unit configured to operate the apparatus. The RF electrodes are organized in non-symmetrical clusters of electrodes. The apparatus operates/addresses each of the RF electrodes individually or entire cluster of the RF electrodes. The control unit is configured to set the duration of an RF energy to skin application, amplitude of the RF waves sufficient to energy application to ablate cylindrical volumes of the tissue under the electrodes, and a sequence of the operation of the matrix RF electrodes. The apparatus supports addressing of individual RF electrodes configured to generate desired areas of lesions. The orientation of the desired areas of lesions supports skin tightening into a desired direction.

The apparatuses control unit is configured to operate the RF generator after the required skin stretching is achieved. The RF generator could be an add-on board located in the control unit.

### LIST OF DRAWINGS AND THEIR SHORT DESCRIPTION

The present method and apparatus will be understood and appreciated from the following detailed description, taken in conjunction with the drawings and wherein like reference numerals denote like elements.

FIG. 1 is a block diagram of the present apparatus for skin tightening;

FIG. 2 is a bottom view of the RF electrodes and the substrate on which the electrodes are mounted;

FIG. 3 is a schematic cross-section of an applicator of the present apparatus;

FIG. 4A is a schematic illustration of the present method of skin tightening;

FIG. 4B is an additional schematic illustration of the present method of skin tightening; and

FIG. 4C is still an additional schematic illustration of the present method of skin tightening.

### DESCRIPTION

Loose skin and wrinkles develop with age or as a result of overexposure to the sun and harsh weather. Sagging skin and fine lines may make you feel like you look older than you are.

The use of RF fractional technology in skin tightening is continuously expanding and becoming a de-facto standard in tightening the skin on the face, neck, or body, and scars masking. The technology utilizes single or multiple RF electrodes that, when applied to the skin and energized, generate a plurality of skin lesions. The skin lesions are also shallow and affect the dermis and sub-dermis skin primarily.

The existing method of use of the technology does not support the directional tightening of the skin. Directional skin tightening means that in one direction, the skin could be tightened more than in another direction.

Stretching the skin beyond normal expansion promotes collagen synthesis and growth. As a result, the skin becomes more elastic, the surface area of the affected skin segment increases, and the skin becomes tighter.

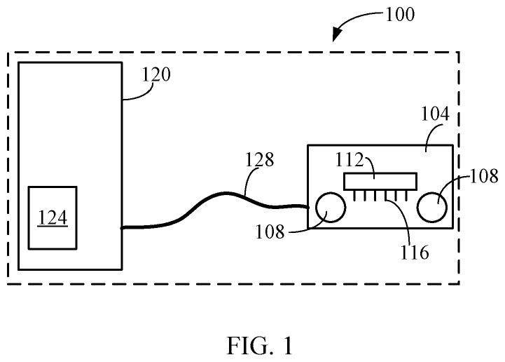

FIG. 1 is a block diagram of the present apparatus for skin tightening. The present apparatus 100 (FIG. 1) includes an applicator 104 with a skin stretching device 108 and a substrate 112 with a two-dimensional array of needle-like RF electrodes 116. The RF electrodes 116 are cylindrical pins or needles with a round or square cross-section. The skin lesions generated by such RF electrodes have a cross-section similar to the needles or pins cross-section. Apparatus 100 further includes a control unit 120, which is an RTC (Real-Time Controller). Control unit 120 includes a GUI (Graphical User Interface), providing the user or caregiver with the possibility to set some treatment parameters. Control unit 120 controls the operation of apparatus 100, sets the operation mode of RF electrodes 116, sets the duration and amplitude of an RF energy application, a sequence of the RF electrodes operation, regulates the skin stretching force, and other processes. An umbilical cable 128 connects between the control unit 120 and applicator 104.

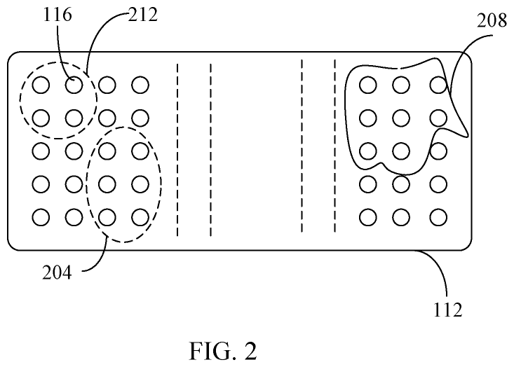

The apparatus includes a two-dimensional array or matrix of RF electrodes 116 arranged on a substrate 112 (FIG. 2) and located in applicator 104. In one example, the RF electrodes 116 are individually addressable. In another example, the RF electrodes 116 are arranged in addressable clusters, for example, clusters 204, 208, and 212. The clusters of the RF electrodes 116 could be symmetrical clusters like cluster 204 or non-symmetrical clusters 208 of RF electrodes 116. Some RF electrodes 116 clusters could include four or five RF electrodes 116, and other clusters could include 10 to 20 RF electrodes 116. In a bipolar mode of operation, some RF electrodes 116 or clusters operate as one of the poles. Another cluster of electrodes or the remaining RF electrodes serves as a returned path electrode.

The individually addressable or operative RF electrodes 116 support the generation or creation of skin lesion patterns of the desired contour and orientation of areas of lesions. The orientation of the areas of lesions supports skin tightening into the desired direction. For example, the operation of (energizing the RF electrodes) clusters 204 and 212 would tighten skin in direction different from the operation of clusters 208 and 208.

In another example, even though the distance between the RF electrodes 116 is symmetrical in both X and Y directions, it is possible to operate in one of the direction every second or third RF electrode 116. Such mode of operation of RF electrodes would define the preferable direction of the skin tightening force.

The RF electrodes 116 could be needle or pin electrodes with a length of 1 to 4 mm. The distance between RF electrodes 116 of the matrix could be 0.25 to 2.5 mm. The cross-section surface of the RF electrodes is 0.1 to 4.0 sq. mm. The number of RF electrodes on the substrate could be 20 to 200 RF electrodes.

Control unit 120 is configured to set the duration and amplitude of the RF energy application and sequence of the matrix RF electrodes' 116 operation. The duration and the amplitude of the RF applied to an RF electrode are selected to ablate in the skin a cylindrical volume of the tissue under the active electrodes at a depth of 0.5 to 3.0 mm.

The RF generator 124 could be an add-on board located in the control unit or console 120 (FIG. 1) or a separate box. In some examples RF generator 124 could be located in applicator 104. Control unit 120 is configured to operate the RF generator 124 after the required skin stretching value is achieved. The RF generator 124 generates RF waves with a frequency of 0.2 MHz to 2.0 MHz and more frequently with a frequency of 0.4 MHz to 0.6 MHz. Control unit 120 is configured to energize individual RF electrodes 116 and clusters or RF electrodes for 3 to 40 milliseconds or any intermediate time value. A complete cycle of energizing all RF electrodes could vary from 0.6 to 2.0 sec and usually 1.0 sec. The control unit is also configured to set a value of an RF energy application to ablate cylindrical skin or tissue volumes below the electrodes 116. RF generator 124 generates RF waves with an amplitude of 100 to 200 volt RMS.

The control unit 120 controlling operation of the RF generator is configured to energize the plurality of RF electrodes 116 according to a predefined sequence. The predefined sequence for energizing the plurality of RF electrodes could include different time intervals.

Skin-stretching devices are known in different medical and cosmetical discipline fields. The present application makes use of a non-invasive skin stretching device 108 built-in into applicator 104, where two rollers, 304 and 308, with a surface coated by a high friction coating are in contact with a treated segment of skin 312 and rotate in different directions. For example, one of the rollers can rotate clockwise, and the other one can rotate counter-clockwise. The friction of the rollers 304 and 308 with the skin applies to segment of skin 312, a force that stretches the segment of skin 312 located between the rollers 304 and 308. Arrow 316 illustrates schematically direction of the force that stretches segment of skin 312. Control unit 120 (FIG. 1) sets the desired tension of the treated segment of skin 312.

Ablative fractional skin treatments produces thousands of very small lesions or wounds to damage a fraction of the skin. The lesions are usually symmetrical to the lesion axes. Healing of these small lesions causes skin tightening. The healing time of each lesion is proportional to the size of the lesion. The present method suggests the formation of asymmetric skin lesions that heal faster in at least one direction and cause a directional skin tightening.

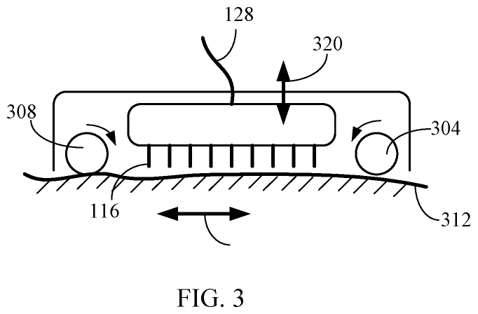

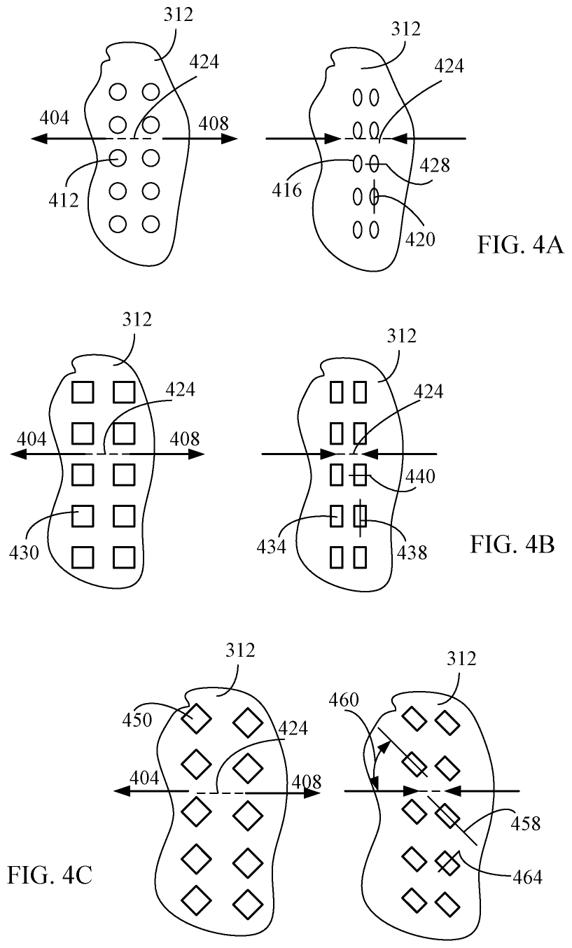

FIG. 4A is a schematic illustration of the present method of skin tightening. The suggested method of skin tightening includes the use of applicator 108 (FIG. 1) that, due to friction with the skin, applies a force for stretching a skin segment 312 in the first direction. The skin segment stretching is between 10 to 35% of the non-stretched skin segment length. When the skin segment is properly stretched, the matrix of RF electrodes 116, as shown by arrow 320, is brought into contact with the skin segment 312 (FIG. 3), and at least one RF pulse is applied to the stretched skin segment 312. The RF pulse applied to the skin segment 312 ablates in the stretched skin segment a plurality of cylindrical lesions 412 with a symmetrical cross-section (FIG. 4A). The location of the plurality of the ablated skin or lesions corresponds to locations of the matrix of electrodes 116. The cross-section of the lesion could be round or square, or rectangular cross-section, similar to the shape of the RF electrode. The depth of the ablated cylindrical skin lesion could be 0.5 to 3.0 mm. The ablated lesion or skin volume could vary from 0.05 mm³ to 4 mm³.

Following the skin lesions 412 formations, the force 404, 408 applied to stretch the skin segment is released. Release of the stretch applied to the skin segment 312 allows the skin segment 312 to return in its non-stretched or initial state. Upon returning the stretched skin segment to its non-stretched state, lesion 412 transforms its symmetrical cross-section shape to an elongated and asymmetrical cross-section shape 416. The long axis 420 of the elongated cross-section shape 416 of the cylindrical lesion is perpendicular to the skin stretching direction 424. The short axis 428 of the elongated cross-section shape 416 of the cylindrical lesion coincides or parallel to the skin stretching direction 424. The lesion 416 healing process in the direction of short-axis 428 of elongated lesion 416 is faster than in the direction of the long elongated lesion axis 420.

FIG. 4B is an additional schematic illustration of the present method of skin tightening. Lesions 430 have a square cross-section. Upon the release of the stretch, lesions 430 transform their symmetrical square cross-section shape into lesions 434 with a rectangular or asymmetric cross-section 434. The long axis 438 of the elongated rectangular cross-section 434 is perpendicular to the skin stretching direction 424. The short axis 440 of the rectangular lesion cross-section shape 434 lesion coincides or parallel to the skin stretching direction 424. The lesion 434 healing process in the direction of short axis 440 of rectangular lesion 434 is faster than in the direction of the long elongated lesion axis 438.

FIG. 4C is still an additional schematic illustration of the present method of skin tightening. Lesions 450 have a square cross-section rotated at an angle to the direction 424 of the skin stretching force. Upon the release of the stretch, lesions 450 transform their symmetrical square cross-section shape into lesions 454 with a rectangular and asymmetric cross-section 454. The long axis 458 of the rotated elongated rectangular cross-section lesion 454 is oriented at an angle 460 to skin stretching direction 424. The short axis 464 of the rectangular lesion cross-section shape 434 lesion is also oriented at an angle 460 +/− 90 degrees to the skin stretching direction 424. The lesion 454 healing process in the direction of short axis 464 of the rotated rectangular lesion 454 is faster than in the direction of the long elongated lesion axis 458.

Several examples have been described. Nevertheless, it will be understood that various modifications may be made without departing from the disclosed method and device's spirit and scope, and method of use. Accordingly, other examples are within the scope of the following claims.

I claim:

1. A method of skin tightening, comprising:
stretching a segment of the skin in a first direction;
applying to the stretched segment of skin at least one RF pulse;
ablating at least one RF ablated skin lesion;
releasing the stretch of the stretched skin segment;
allowing the skin to return in its non-stretched state; and
wherein upon return of the stretched skin segment to its non-stretched state, the lesion transforms its symmetrical cross-section shape to an elongated cross-section shape.

2. The method of claim 1, wherein the at least one RF pulse generates in the stretched segment of skin a cylindrical lesion with a symmetric cross-section.

3. The method of claim 1, wherein a long axis of an elongated cross-section shape of a cylindrical lesion is perpendicular to skin stretching direction.

4. The method of claim 1, wherein the at least one RF pulse is one of a group of pulses applied by a monopolar or a bi-polar matrix of RF electrodes.

5. The method of claim 1, wherein healing of the elongated lesion in the direction of short elongated lesion axes is faster than in the direction of a long elongated lesion axis.

6. The method of claim 1, wherein a control unit is configured to energize a plurality of RF electrodes according to a predefined sequence.

7. The method of claim 1, wherein a control unit is configured to energize individual RF electrodes and clusters for 3 to 40 milliseconds.

8. The method of claim 1, wherein operation of different RF electrodes clusters provides skin tightening in different directions.

9. The method of claim 1, wherein an ablated cylindrical skin lesion has a depth of 0.5 to 3.0 mm.

10. The method of claim 1, wherein a control unit is configured to set a value of an RF energy application to ablate cylindrical volumes below the RF electrodes.

11. The method of claim 1, wherein the skin segment stretching is between 10 to 35% of a non-stretched skin segment length.

12. A method of skin tightening, comprising:
applying a force to stretch a segment of skin in a first direction;
applying to the stretched segment of skin a matrix of RF electrodes and at least one RF pulse;
ablating a plurality of skin locations corresponding to locations of the matrix of electrodes to form a plurality of RF ablated lesions;
releasing the stretch of the stretched skin segment;
allowing the skin to return in its non-stretched state; and
and wherein the ablated skin lesion has a depth of 0.5 to 3.0 mm.

13. The method of claim 12, wherein the RF ablated skin lesions have a symmetric cross-section shape.

14. The method of claim 13, wherein upon release of the stretch of the skin segment the symmetric cross-section shape lesions transform its shape to an asymmetric shape lesions.

15. The method of claim 12, wherein the method supports addressing of individual RF electrodes configured to generate desired areas of lesions.

16. The method of claim 12, wherein orientation of desired areas of lesions supports skin tightening into a desired direction.

17. A method of skin treatment, comprising:
applying to a segment of skin a skin stretching device and stretching the segment of skin in a first direction;
applying to the stretched segment of skin a matrix of RF electrodes and delivering at least one RF pulse;
ablating the stretched skin segment to form a matrix of RF ablated symmetrical skin lesions;
releasing the stretch of the stretched skin segment and allowing the skin to return in its non-stretched state; and
wherein upon release of the stretch the symmetrical skin lesions transform their shape into elongated in a direction perpendicular to the skin stretching direction skin lesions.

18. An apparatus for skin treatment/tightening, comprising:
a matrix of RF electrodes disposed on a rigid or flexible substrate;
an RF generator configured to energize the RF electrodes;
a device configured to stretch the treated segment of skin; and
a control unit configured to operate the device.

19. The apparatus of claim 18, wherein the RF electrodes are at least one of a group of RF electrodes, including bipolar and monopolar RF electrodes.

20. The apparatus of claim 19 wherein the RF electrodes are organized in non-symmetrical clusters of electrodes.

21. The apparatus of claim 18, wherein a distance between the RF electrodes of the matrix is 0.25 to 2.5 mm.

22. The apparatus of claim 18, wherein the control unit, is configured to set duration of an RF energy application and a sequence of the operation of the matrix RF electrodes.

23. The apparatus of claim 22, wherein the control unit is configured to set duration of an RF energy application to ablate cylindrical volumes of the tissue under the electrodes

24. The apparatus of claim 22, wherein the control unit is configured to operate the RF generator after the required skin stretching is achieved.

25. The apparatus of claim 18, wherein the RF generator computer is an add-on board located in the control unit.

26. The apparatus of claim 25, wherein the RF generator generates RF waves with a frequency of 0.2 to 2.0 MHz.

27. The apparatus of claim 25, wherein the RF generator generates RF waves with a 100-200 volt RMS amplitude.

28. The apparatus of claim 18, wherein the apparatus supports addressing of individual RF electrodes configured to generate desired areas of lesions.

29. The apparatus of claim 28, wherein orientation of the desired areas of lesions supports skin tightening into the desired direction.
